(19) United States
(12) Patent Application Publication
(10) Pub. No.: US 2008/0227971 A1
(43) Pub. Date: Sep. 18, 2008

(54) Deacylation of sucralose-6-acylates

(76) Inventors: Duane A. Leinhos, Satsuma, AL (US); David A. Dentel, Jurong Island (SG); James Edwin Wiley, Daphne, AL (US)

Correspondence Address: RATNERPRESTIA, P.O. BOX 1596, WILMINGTON, DE, 19899, US

(21) Appl. No.: 12/004,260

(22) Filed: Dec. 20, 2007

Related U.S. Application Data
(60) Provisional application No. 60/881,292, filed on Jan. 19, 2007.

(51) Int. Cl. C07H 1/06

(52) U.S. Cl. 536/124

(57) ABSTRACT

A method of making sucralose includes maintaining a solution of a sucralose-6-acylate in an aqueous solvent at a temperature in a range from −20° C. to 20° C. and at a pH of at least 12.2 and less than 14.0 for a period of time sufficient to deacylate substantially all of the sucralose-6-acylate. The method may employ purified sucralose-6-acylate as the starting material, or the sucralose-6-acylate may be present in a quenched reaction mixture resulting from chlorination of a sucrose-6-acylate. The quenched reaction mixture may be exposed to deacylation conditions either before or after removal of a tertiary amide that may be present in it. Sucralose is subsequently recovered to complete the process.

### CROSS REFERENCE TO RELATED APPLICATIONS

This application claims priority benefit of U.S. provisional patent application No. 60/881,292, filed Jan. 19, 2007, the entire disclosure of which is expressly incorporated herein by reference.

### BACKGROUND OF THE INVENTION

Sucralose (4,1′,6′-trichloro-4,1′,6′-trideoxygalactosucrose), a high-intensity sweetener made from sucrose, can be used in many food and beverage applications.

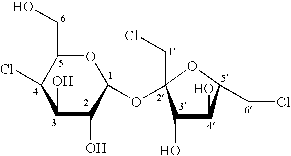

A number of different synthetic routes for the preparation of sucralose have been developed in which the reactive hydroxyl in the 6 position is first blocked with an acyl group to form a sucrose-6-acylate. The acyl group is typically acetyl or benzoyl, although others may be used. The sucrose-6-acylate is then chlorinated to replace the hydroxyls at the 4, 1′ and 6′ positions to produce 4,1′,6′-trichloro-4,1′,6′-trideoxygalactosucrose 6-acylate (referred to herein as sucralose-6-acylate), followed by hydrolysis to remove the acyl substituent and thereby produce sucralose. Several synthesis routes for formation of the sucrose-6-acylates involve tin-mediated acylation reactions, with illustrative examples being disclosed in U.S. Pat. Nos. 4,950,746; 5,023,329; 5,089,608; 5,034,551; and 5,470,969, all of which are incorporated herein by reference.

Various chlorinating agents may be used to chlorinate the sucrose-6-acylate, and most commonly a Vilsmeier-type salt such as Arnold's Reagent will be used. One suitable chlorination process is disclosed by Walkup et al. (U.S. Pat. No. 4,980,463), in which a tertiary amide, typically N,N-dimethylformamide (“DMF”), is used as the chlorination reaction solvent. After the chlorination is complete, the excess chlorinating reagent is neutralized (“quenched”) with aqueous base to provide the sucralose-6-acylate in an aqueous solution, accompanied by the tertiary amide solvent and salts resulting from reactions of the chlorination reagent.

The sucralose-6-acylate is then deacylated to produce sucralose. One suitable process is taught by Navia et al, U.S. Pat. No. 5,498,709, the entire disclosure of which is incorporated herein by reference. The method of Navia involves deacylating the sucralose-6-acylate without first isolating it. This may be done before removing DMF from the reaction mixture (typically by steam stripping), but Navia prefers to perform this step after DMF removal to avoid hydrolysis of DMF under the conditions of deacylation, which include raising the pH of the sucralose-6-acylate solution to a value of 11±1. While the deacylation methods disclosed by Navia are generally effective, there is opportunity for improving the yield of sucralose produced by deacylation of sucralose-6-acylates. Further, the ability to deacylate in the presence of DMF without excessive hydrolysis would be advantageous in some methods of making sucralose.

### SUMMARY OF THE INVENTION

In one aspect, the invention provides a method of making sucralose, including

a) maintaining a solution including a sucralose-6-acylate in an aqueous solvent at a temperature in a range from −20° C. to 20° C. and at a pH of at least 12.2 and less than 14.0 for a period of time sufficient to deacylate substantially all of the sucralose-6-acylate; and

b) subsequently recovering the sucralose.

In another aspect, the invention provides a method of making sucralose, including

a) maintaining a solution including sucralose-6-acetate in an aqueous solvent at a temperature in a range from −20° C. to 20° C. and at a pH of at least 12.2 and less than 14.0 for a period of time sufficient to deacylate substantially all of the sucralose-6-acetate; and

b) subsequently recovering the sucralose.

In yet another aspect, the invention provides a method of making sucralose, including

a) maintaining a solution including sucralose-6-benzoate in an aqueous solvent at a temperature in a range from −20° C. to 20° C. and at a pH of at least 12.2 and less than 14.0 for a period of time sufficient to deacylate substantially all of the sucralose-6-benzoate; and

b) subsequently recovering the sucralose.

### BRIEF DESCRIPTION OF THE DRAWINGS

FIG. 1 shows deacylation reaction progress as a function of time under a variety of reaction conditions according to the invention, compared with deacylation reactions performed under prior art conditions.

FIG. 2 shows the amounts of 3′,6′-anhydro-4,1′-dichlorogalactosucrose formed at various times during the deacylation reactions depicted in FIG. 1.

### DETAILED DESCRIPTION OF THE INVENTION

The applicants has found that improved sucralose yields may be obtained when sucralose-6-acylate is deacylated in a pH range higher than that disclosed in Navia and other prior art. Specifically, a pH value of at least 12.2 and less than 14.0 may be used if the deacylation is performed at a sufficiently low temperature, while still retaining satisfactory reaction rates. Moreover, use of these conditions makes it possible to perform deacylation with very little hydrolysis of DMF, thus making it practical to deacylate prior to removing the DMF. This carries significant advantages, for reasons that will now be described.

Sucralose-6-acylates are quite soluble in mixtures of DMF and water, but their solubility in water alone is limited. Thus, if DMF is stripped prior to deacylation as traditionally practiced, the applicant has found that a significant quantity of solids (some of which is the solid sucralose-6-acylate itself) fouls the steam stripper during removal of DMF from the quenched chlorination mixture. In addition to creating an equipment maintenance problem, this may in some cases potentially reduce sucralose yield because the solids are difficult to recycle into product.

In light of this limited solubility and tendency to foul reactors and create a yield loss, one might consider deacylating a sucralose-6-acylate prior to stripping the DMF, since the deacylated product is of higher solubility than the acylate in the remaining solvent (water). However, this has traditionally been considered an undesirable way of operating, due to an expectation that too much DMF will be hydrolyzed under the conditions needed to effect deacylation. For example, U.S. Pat. No. 5,498,709 to Navia et al points to this problem and concludes that, although deacylation can be performed in the presence of DMF, it is not preferred for this very reason.

Despite these teachings, the applicant has now found that, by deacylating at a pH of at least 12.2 and less than 14.0 at sufficiently low temperatures, the reaction may be performed in the presence of tertiary amides such as DMF with only very little hydrolysis of the amide. What is more, this method can produce sucralose yields exceeding those obtained under the pH conditions taught by Navia et al, i.e., pH values not exceeding 12.

As noted above, steam stripping of DMF from sucralose-6-acylate solutions produces insoluble materials that adhere to processing vessels as the solubilizing effect of DMF is removed. In contrast, the applicant has found that solids produced upon stripping DMF after deacylation show a markedly lower tendency to adhere to processing equipment.

The applicant has also found another benefit of deacylating under the presently disclosed pH and temperature conditions prior to DMF stripping, namely that dimethylamino dimethyl ammonium chloride (DMAM), an impurity commonly present in the mixture, is typically hydrolyzed under the deacylation conditions of this invention. The applicant has found that the products of this hydrolysis (DMF and dimethylamine) may be effectively removed during subsequent steam stripping of DMF. In contrast, if deacylation is performed after DMF stripping (as preferred by Navia), DMF and dimethylamine are subsequently generated. This is of course undesirable because it requires an additional purification step to remove these later-formed impurities.

Sucralose-6-acylate from any source may be deacylated according to the invention. It may optionally be purified and isolated prior to the deacylation, and then dissolved in either water or a water/solvent mixture such as water/DMF. Typically, the sucralose-6-acylate will be present in a quenched reaction mixture resulting from chlorination of sucrose-6-acylate, for example as described by U.S. Pat. No. 4,980,463, incorporated herein by reference. The quenched reaction mixture is typically an aqueous mixture, by which it is meant that water is the largest single constituent of the solvent by weight. Typically, a tertiary amide such as DMF is the next largest component of the solvent, and it generally constitutes at least 15 wt % of the solvent, more typically at least 25 wt % of the solvent. In many cases, it is as high as 35 or 40 wt % of the solvent, although it may be even more. The composition of is one exemplary quenched chlorination reaction mixture is as shown in Table 1, along with typical ranges of values for the components according to three embodiments of the invention. The components are listed as wt % values based on the total mixture.

TABLE 1

| | Ex- | First | Second | Third |
|---|---|---|---|---|
| Component | ample | Embodiment | Embodiment | Embodiment |
| Water | 49 | 30-45 | 15-85 | 10-92 |
| DMF | 32 | 30-40 | 20-55 | 15-65 |
| Sodium chloride | 8 | 10-14 | 5-25 | 3-30 |
| DMAM | 4 | 1-3 | 0.4-5 | 0.1-6 |
| Sucralose-6-acylate | 3 | 2-3 | 1-5 | 0.5-7 |
| Sodium acetate | 1 | 0.5-1.5 | 0.2-2 | 0.1-3 |
| Others | 3 | 2-4 | 1-6 | 0.5-8 |

In some embodiments of the invention, the quenched reaction mixture is brought to a pH of at least 12.2 and less than 14.0 without first stripping the DMF, and maintained at a temperature in a range from −20° C. to 20° C. for a period of time sufficient to deacylate substantially all (i.e., at least 95%) of the sucralose-6-acylate. The pH of the reaction mixture may suitably be raised to the desired value by the addition of alkali metal hydroxides such as NaOH. The lower limit on pH will typically be 12.2, and in some cases will be 12.3 or 12.4. The upper limit will typically be 13.8, and in some cases it will be 13.6 or 13.4. In some preferred embodiments, the pH will be at most 13.0. For example, the pH may be in a range from 12.2 to 13.8, or in a range from 12.4 to 13.0.

Suitable temperatures for the deacylation are at least −20° C., more typically at least −10° C., and most typically at least 0° C. The practical lower temperature limit will of course be affected by the exact amount and type of tertiary amide (if present) and the amount and type of sucralose-6-acylate, and it is preferable that the solvent remain substantially unfrozen and the sucralose-6-acylate remain substantially in solution during the deacylation. Suitable upper limits for the deacylation temperature are at most 20° C., more typically no higher than 15° C., and most typically at most 10° C. For example, the temperature may be in a range from −5° C. to 10° C. or from 0° C. to 15° C.

In general, the applicant has found that deacylations at higher pH values work best at relatively lower temperatures, and lower pH values work best at relatively higher temperatures. Accordingly, in some embodiments of the invention the temperature is selected according to the following equation:

T(° C.)=(−14.724)(pH)+193.52±5 (or ±2, in some embodiments)

Exemplary temperature ranges according to the foregoing equation are shown in Table 2.

TABLE 2

Preferred

| | Upper | Lower | Upper | Lower |
|---|---|---|---|---|
| pH | Temp | Temp | Temp | Temp |
| 12.2 | 18.9 | 8.9 | 15.9 | 11.9 |
| 12.3 | 17.4 | 7.4 | 14.4 | 10.4 |
| 12.4 | 15.9 | 5.9 | 12.9 | 8.9 |
| 12.5 | 14.5 | 4.5 | 11.5 | 7.5 |
| 12.6 | 13.0 | 3.0 | 10.0 | 6.0 |
| 12.7 | 11.5 | 1.5 | 8.5 | 4.5 |
| 12.8 | 10.1 | 0.1 | 7.1 | 3.1 |
| 12.9 | 8.6 | −1.4 | 5.6 | 1.6 |
| 13.0 | 7.1 | −2.9 | 4.1 | 0.1 |
| 13.1 | 5.6 | −4.4 | 2.6 | −1.4 |
| 13.2 | 4.2 | −5.8 | 1.2 | −2.8 |
| 13.3 | 2.7 | −7.3 | −0.3 | −4.3 |
| 13.4 | 1.2 | −8.8 | −1.8 | −5.8 |
| 13.5 | −0.3 | −10.3 | −3.3 | −7.3 |
| 13.6 | −1.7 | −11.7 | −4.7 | −8.7 |
| 13.7 | −3.2 | −13.2 | −6.2 | −10.2 |
| 13.8 | −4.7 | −14.7 | −7.7 | −11.7 |
| 13.9 | −6.1 | −16.1 | −9.1 | −13.1 |
| 14.0 | −7.6 | −17.6 | −10.6 | −14.6 |

Reaction times to provide substantially complete deacylation range considerably, depending inter alia on the pH, the temperature, and the particular acyl group on the sucralose-6-acylate (for example acetyl or benzoyl, although others may be used according to the invention). Typically, the reaction time will be in a range from 0.5 to 8 hours, and more typically in a range from 1 to 6 hours. However, longer or shorter times may be used. Progress of the deacylation may be monitored by high performance liquid chromatography (HPLC), and when the reaction is complete the pH is reduced by the addition of a suitable acid such as hydrochloric acid, citric acid, or acetic acid to stop the reaction. Usually, the final pH value is in a range from 6 to 8.5, typically about 7.5.

The solvent is then stripped. This can be performed by any means known in the art, such as distillation under atmospheric or reduced pressure, steam distillation, steam stripping, or by use of an agitated thin film drier or spray drier. Typically, steam stripping is performed to remove tertiary amide solvents such as DMF, for example according to the methods disclosed in U.S. Pat. No. 5,498,709. Final isolation of sucralose can be carried out by any known means, for example the methods disclosed in that same patent.

Although there are many advantages to deacylating sucralose-6-acylates under the above-mentioned pH and temperature conditions before stripping the tertiary amide solvent, deacylation may also be performed after such stripping according to some embodiments of the invention. Side reactions to form 3′,6′-anhydro-4,1′-dichlorogalactosucrose tend to be relatively slow under the pH and temperature conditions of this invention. This advantage remains regardless of whether the tertiary amide is stripped before or after deacylation, since it relates to reducing side reactions of the sucralose and sucralose precursors themselves.

If the tertiary amide is stripped prior to deacylation, such stripping is typically performed until the tertiary amide constitutes at most about 10 wt % of the total mixture. Typically it constitutes 6-10 wt % to help maintain solubility of the sucralose-6-acylate. After deacylation, stripping is typically performed again to a DMF level of less than 0.1 wt % to avoid interference with crystallization of the product. Regardless of whether the deacylation is performed before or after the tertiary amide is removed, the final sucralose product is ultimately isolated and purified by crystallization, chromatography, and/or extraction techniques such as are known in the art.

### EXAMPLES

Each of the following examples used, as starting material, quenched chlorination material obtained by the following method, described generally in U.S. Pat. No. 5,498,709 to Navia. Phosgene was added to a solution of sucrose-6-acetate in DMF in an amount sufficient to convert essentially all of the 7 hydroxyl groups to adducts with Arnold's reagent. The mixture was heated to effect chlorination at the 4, 1′ and 6′ positions, and then cooled and quenched with aqueous NaOH to a pH of 9.

In each Example, quenched chlorination product as described above was charged to a temperature controlled, agitated vessel. The pH was measured by direct immersion of a Thermo Orion 916200 electrode coupled with Eutech pH 800 controller, pH equipment calibrated with standard buffers at pH 7.0 and 10.0 using temperature compensation. The apparatus was capable of mixing a solution of quenched sucralose-6-acylate at a defined and maintained temperature at a selected pH. Caustic (2-50%) and acid (2-18N) was pumped as needed into the vessel via pumps controlled by a pH controller. Temperature was maintained by a constant temperature control, and analyses of reaction mixtures were performed by high performance liquid chromatography (HPLC) and/or infrared analysis.

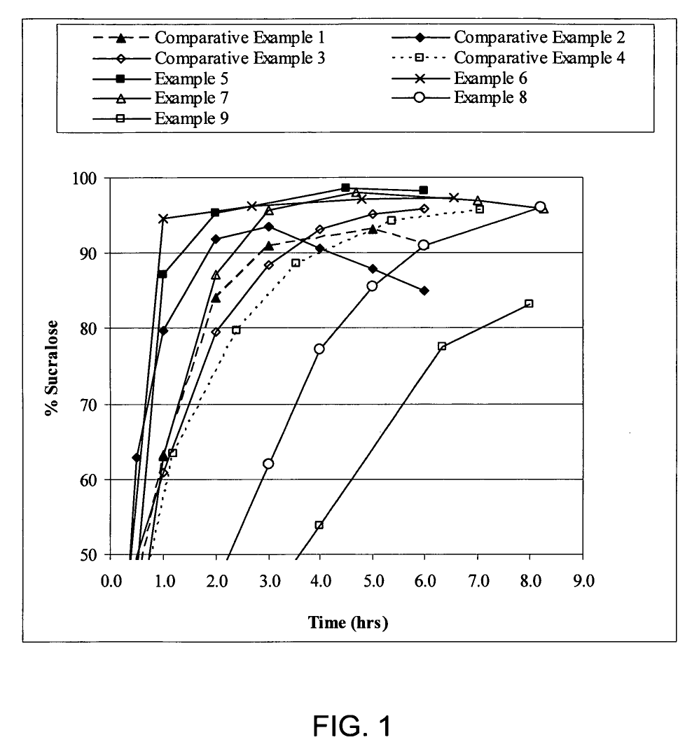

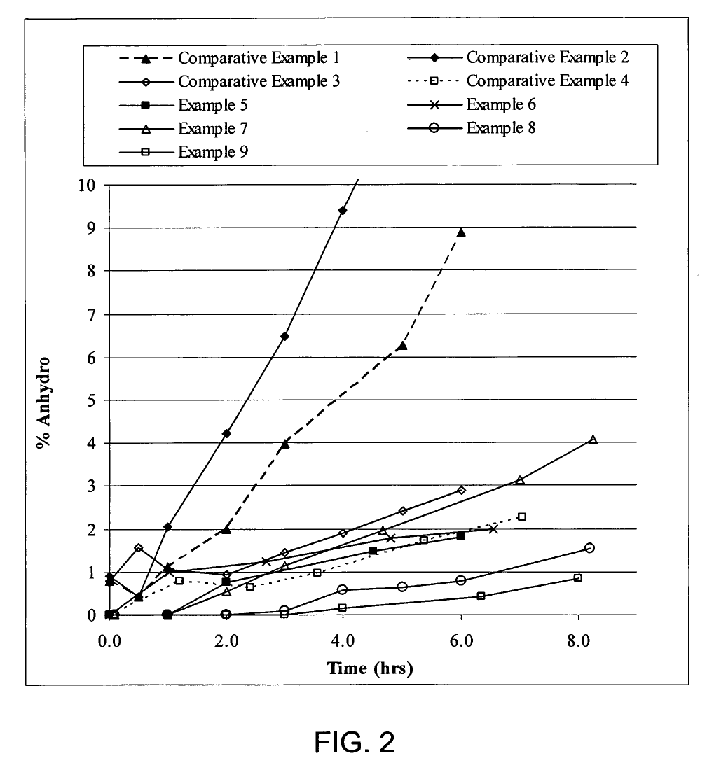

It is useful to express deacylation performance in terms of the amount of desired product (e.g. sucralose) as a percentage of the total of sucralose, degraded sucralose (3′,6′-anhydro-4,1′-dichlorogalactosucrose), and sucralose precursor (i.e., unreacted acylate). Thus, the molar percentage of realized desired product divided by potential moles of desired product is a reflection of the ability to obtain a maximum (100%) of conversion to sucralose. A number of examples are provided below, showing the necessary reaction times for deacylating sucralose-6-acetate under various pH and temperature conditions. One would, of course, desire to convert all sucralose-6-acetate to sucralose with no loss of sucralose to 3′,6′-anhydro-4,1′-dichlorogalactosucrose, and an ability to approach this goal would reflect favorably upon the method. Five examples and four comparative examples are provided below. The comparative examples were all run at a pH no greater than 11.8, and the examples according to the invention were all run at a pH of at least 12.2. Yields of sucralose in each case were tracked as a function of time, and are summarized in Table 3 and in FIG. 1 for purposes of comparison. Formation of 3′,6′-anhydro-4,1′-dichlorogalactosucrose was also tracked, and the results are indicated in FIG. 2.

In all examples, sucralose yields were calculated molewise as a percentage of the total moles of sucralose+3′,6′-anhydro-4,1′-dichlorogalactosucrose+sucralose-6-acetate. In FIG. 2, the % Anhydro values were calculated molewise as a percentage of the total moles of sucralose+3′,6′-anhydro-4,1′-dichlorogalactosucrose+sucralose-6-acetate.

A review of FIG. 1 reveals that the conditions that most closely approach 100% of potential sucralose yield included a pH above the 11±1 range disclosed by Navia et al.

As can be seen in FIG. 2, side reactions to form 3′,6′-anhydro-4,1′-dichlorogalactosucrose tended to be slower under the pH and temperature conditions of this patent, compared with those of the prior art.

### Comparative Example 1

A deacetylation reaction was performed above ambient temperature as follows. A 400 g sample of quenched chlorination reaction mixture was brought to a temperature of 40° C. in a jacketed reactor using a constant temperature circulating bath and pH adjusted to 10.5 with 10% NaOH as measured directly by laboratory pH controller. The pH was maintained by periodic addition of 10% NaOH via pump under the control of the pH controller. Samples, and the final reaction mixture, were lowered to a pH of 7.5 by addition of either concentrated acetic acid or concentrated hydrochloric acid to stop the deacylation reaction. Maximum yield was realized at 5 hours with 93.1% sucralose formation.

### Comparative Example 2

A 400 g sample of quenched chlorination reaction mixture was brought to 40° C. and pH adjusted to 10.7 with addition of 10% NaOH. The temperature was maintained under these conditions with addition of NaOH until the reaction was stopped by addition of concentrated acid. Maximum yield was realized at 3 hours with 93.4% sucralose formation.

### Comparative Example 3

A 400 g sample of quenched chlorination reaction mixture was brought to 27° C. and pH adjusted to 11.2 with addition of 10% NaOH. Temperature was maintained by a constant temperature circulating bath with flow through the reactor jacket and pH maintained by a pH controller pumping in additional NaOH. The reaction was stopped by addition of concentrated acetic or hydrochloric acid. Maximum yield was realized at 6 hours with an improved 95.8% sucralose formation.

### Comparative Example 4

A 600 g sample of quenched chlorination reaction mixture was maintained at 20° C. and adjusted to pH 11.8 with 10% NaOH. These pH and temperature conditions were maintained, using a pH meter and periodic manual pH adjustment, until the deacylation reaction was stopped by addition of concentrated hydrochloric acid. Maximum yield was realized at 7 hours with 95.7% sucralose formation. This experiment incorporated vacuum removal of dimethylamine.

### Example 5

A 503 g sample of quenched chlorination reaction mixture was maintained at 11° C. and adjusted to pH 12.4 with 10% NaOH. These pH and temperature conditions were maintained until the deacylation reaction was stopped by adjusting pH to 7.5 with concentrated acetic acid. Maximum yield was realized at 4.5 hours with 98.5% sucralose formation.

### Example 6

A 600 g sample of quenched chlorination reaction mixture was maintained at 11° C. and adjusted to pH 12.4 with 15% NaOH. These pH and temperature conditions were maintained until the deacylation reaction was stopped by adjusting pH to 7.5 with concentrated hydrochloric acid. Maximum yield was realized at 6.5 hours with 97.24% sucralose formation. This experiment incorporated vacuum removal of dimethylamine.

### Example 7

A 400 g sample of quenched chlorination reaction mixture was maintained at 10° C. and adjusted to pH 12.5 with 10% NaOH. These pH and temperature conditions were maintained until the deacylation reaction was stopped by addition of concentrated hydrochloric acid. Maximum yield was realized at 4.7 hours with 98.0% sucralose formation.

### Example 8

A 400 g sample of quenched chlorination reaction mixture was maintained at 10° C. and adjusted to pH 12.35 with 10% NaOH. These pH and temperature conditions were maintained until the deacylation reaction was stopped by addition of concentrated hydrochloric acid. At 8.2 hours reaction time, sucralose yield had reached 96.0% and was still climbing.

### Example 9

A 400 g sample of quenched chlorination reaction mixture was maintained at 10° C. and adjusted to pH 12.2 with 10% NaOH. These pH and temperature conditions were maintained until the deacylation reaction was stopped by addition of concentrated hydrochloric acid. At 8 hours reaction time, sucralose yield had reached 83.2% and was still climbing. The rate of this reaction was somewhat slow, consistent with the fact that the temperature was considerably below an expected ideal temperature for a pH of 12.2 of about 14° C., as indicated by the previously-noted relationship T(° C.)=(−14.724)(pH)+193.52.

TABLE 3

| | | Temp. | Max. |
|---|---|---|---|
| Run | pH | (° C.) | Yield (%) |
| Comparative Example 1 | 10.5 | 40 | 93.1 |
| Comparative Example 2 | 10.7 | 40 | 93.4 |
| Comparative Example 3 | 11.2 | 27 | 95.8 |
| Comparative Example 4 | 11.8 | 20 | 95.7 |
| Example 5 | 12.4 | 11 | 98.5 |
| Example 6 | 12.4 | 11 | 97.2 |
| Example 7 | 12.5 | 10 | 98.0 |
| Example 8 | 12.35 | 10 | *NR |
| Example 9 | 12.2 | 10 | *NR |

*Not Reached

As can be seen from the foregoing Examples and Comparative examples, increased sucralose yields may be realized using the methods of this invention. Although the invention is illustrated and described herein with reference to specific embodiments, the invention is not intended to be limited to the details shown. Rather, various modifications may be made in the details within the scope and range of equivalents of the claims without departing from the invention.

What is claimed:

1. A method of making sucralose, comprising
a) maintaining a solution comprising a sucralose-6-acylate in an aqueous solvent at a temperature in a range from −20° C. to 20° C. and at a pH of at least 12.2 and less than 14.0 for a period of time sufficient to deacylate substantially all of the sucralose-6-acylate; and
b) subsequently recovering the sucralose.

2. The method of claim 1, wherein the temperature is no higher than 15° C.

3. The method of claim 1, wherein the pH is in a range from 12.3 to 13.8.

4. The method of claim 1, wherein the pH is in a range from 12.4 to 13.6.

5. The method of claim 1, wherein the period of time is in a range from 0.5 to 8 hours.

6. The method of claim 1, wherein the period of time is in a range from 1 to 6 hours.

7. The method of claim 1, wherein the solvent further comprises a tertiary amide.

8. The method of claim 7, wherein the tertiary amide is DMF.

9. The method of claim 7, wherein the tertiary amide constitutes at least 15 wt % of the solvent.

10. The method of claim 7, wherein the tertiary amide constitutes at most 10 wt % of the solvent.

11. The method of claim 1, wherein the sucralose-6-acylate is sucralose-6-acetate.

12. The method of claim 1, wherein the sucralose-6-acylate is sucralose-6-benzoate.

13. The method of claim 1, further comprising preparing the solution comprising the sucralose-6-acylate by a method comprising contacting a solution comprising sucrose-6-acylate in a tertiary amide with a chlorinating reagent under conditions sufficient to form the sucralose-6-acylate, and then contacting the resulting reaction mixture with an aqueous alkaline solution.

14. The method of claim 1, wherein the temperature (° C.) is in a range given by the formula (−14.724)(pH)+193.52±5.

15. A method of making sucralose, comprising
a) maintaining a solution comprising sucralose-6-acetate in an aqueous solvent at a temperature in a range from −20° C. to 20° C. and at a pH of at least 12.2 and less than 14.0 for a period of time sufficient to deacylate substantially all of the sucralose-6-acetate; and
b) subsequently recovering the sucralose.

16. The method of claim 15, wherein the solvent further comprises a tertiary amide.

17. The method of claim 16, wherein the tertiary amide is DMF.

18. The method of claim 16, wherein the tertiary amide constitutes at least 15 wt % of the solvent.

19. The method of claim 16, wherein the tertiary amide constitutes at most 10 wt % of the solvent.

20. The method of claim 15, further comprising preparing the solution comprising the sucralose-6-acetate by a method comprising contacting a solution comprising sucrose-6-acetate in a tertiary amide with a chlorinating reagent under conditions sufficient to form the sucralose-6-acetate, and then contacting the resulting reaction mixture with an aqueous alkaline solution.

21. The method of claim 15, wherein the temperature (° C.) is in a range given by the formula (−14.724)(pH)+193.52±5.

22. A method of making sucralose, comprising
a) maintaining a solution comprising sucralose-6-benzoate in an aqueous solvent at a temperature in a range from −20° C. to 20° C. and at a pH of at least 12.2 and less than 14.0 for a period of time sufficient to deacylate substantially all of the sucralose-6-benzoate; and
b) subsequently recovering the sucralose.

23. The method of claim 22, wherein the solvent further comprises a tertiary amide.

24. The method of claim 23, wherein the tertiary amide is DMF.

25. The method of claim 23, wherein the tertiary amide constitutes at least 15 wt % of the solvent.

26. The method of claim 23, wherein the tertiary amide constitutes at most 10 wt % of the solvent.

27. The method of claim 22, further comprising preparing the solution comprising the sucralose-6-benzoate by a method comprising contacting a solution comprising sucrose-6-benzoate in a tertiary amide with a chlorinating reagent under conditions sufficient to form the sucralose-6-benzoate, and then contacting the resulting reaction mixture with an aqueous alkaline solution.

28. The method of claim 22, wherein the temperature (° C.) is in a range given by the formula (−14.724)(pH)+193.52±5.
